(19) United States
(12) Patent Application Publication
(10) Pub. No.: US 2013/0129901 A1
(43) Pub. Date: May 23, 2013

(54) PRODUCTION OF SOLUBLE PROTEIN SOLUTIONS FROM PULSES

(71) Applicants: Kevin I. Segall, Winnipeg (CA); Martin Schweizer, Winnipeg (CA)

(72) Inventors: Kevin I. Segall, Winnipeg (CA); Martin Schweizer, Winnipeg (CA)

(73) Assignee: BURCON NUTRASCIENCE (MB) CORP., Winnipeg, MB (CA)

(21) Appl. No.: 13/642,003

(22) PCT Filed: May 9, 2011
(86) PCT No.: PCT/CA2011/000529

§ 371 Date: Jan. 7, 2013

Related U.S. Application Data
(60) Provisional application No. 61/344,013, filed on May 7, 2010.

(51) Int. Cl. C07K 1/14

(52) U.S. Cl. CPC C07K 1/145
USPC 426/598; 530/370

(57) ABSTRACT

A pulse protein product, which may be an isolate, produces heat-stable solutions at low pH values and is useful for the fortification of soft drinks and sports drinks without precipitation of protein. The pulse protein product is obtained by extracting a pulse protein source material with an aqueous calcium salt solution to form an aqueous pulse protein solution, separating the aqueous pulse protein solution from residual pulse protein source, adjusting the pH of the aqueous pulse protein solution to a pH of about 1.5 to about 4.4 to produce an acidified pulse protein solution, which may be dried, following optional concentration and diafiltration, to provide the pulse protein product.

### REFERENCE TO RELATED APPLICATION

This application claims priority under 35 USC 119(e) from U.S. Provisional Patent Application No. 61/344,013 filed May 7, 2010.

### FIELD OF INVENTION

The present invention is directed to the production of protein solutions from pulses and to novel pulse protein products.

### BACKGROUND TO THE INVENTION

In U.S. patent application Ser. Nos. 12/603,087 filed Oct. 21, 2009 (US Patent Publication No. 2010-0098818) and 12/923,897 filed Oct. 13, 2010 (US Patent Publication No. 2011-0038993), assigned to the assignee hereof and the disclosures of which are incorporated herein by reference, there is described the production of soy protein products having a protein content of at least about 60 wt % (N×6.25) d.b., preferably at least about 90 wt %, which produce transparent, heat stable solutions at low pH value and which may be used for protein fortification of soft drinks, as well as other aqueous systems, without precipitation of protein.

The soy protein product is produced by extracting a soy protein source with an aqueous calcium chloride solution to cause solubilization of soy protein from the protein source and to form an aqueous soy protein solution, separating the aqueous soy protein solution from residual soy protein source, optionally diluting the soy protein solution, adjusting the pH of the aqueous soy protein solution to a pH of about 1.5 to about 4.4, preferably about 2 to about 4, to produce an acidified clear soy protein solution, optionally concentrating the aqueous clear protein solution while maintaining the ionic strength substantially constant by using a selective membrane technique, optionally diafiltering the concentrated soy protein solution, and optionally drying the concentrated and optionally diafiltered soy protein solution.

### SUMMARY OF THE INVENTION

It has been found that this procedure and modifications thereof, may be used to form acid soluble protein products from pulses, including lentils, chickpeas, dry peas and dry beans.

Accordingly, in one aspect of the present invention, there is provided a method of producing a pulse protein product having a pulse protein content of at least about 60 wt %, preferably at least about 90 wt %, (N×6.25) on a dry weight basis, which comprises:

(a) extracting a pulse protein source with an aqueous calcium salt solution, preferably an aqueous calcium chloride solution, to cause solubilization of pulse protein from the protein source and to form an aqueous pulse protein solution,

(b) separating the aqueous pulse protein solution from residual pulse protein source,

(c) optionally diluting the aqueous pulse protein solution,

(d) adjusting the pH of the aqueous pulse protein solution to a pH of about 1.5 to about 4.4, preferably about 2 to about 4, to produce an acidified pulse protein solution,

(e) optionally clarifying the acidified pulse protein solution if it is not already clear,

(f) alternatively from steps (b) to (e), optionally, diluting and then adjusting the pH of the combined aqueous pulse protein solution and residual pulse protein source to a pH of about 1.5 to about 4.4, preferably about 2 to about 4, then separating the acidified, preferably clear, pulse protein solution from residual pulse protein source,

(g) optionally concentrating the aqueous pulse protein solution while maintaining the ionic strength substantially constant by a selective membrane technique,

(h) optionally diafiltering the concentrated pulse protein solution, and

(i) optionally drying the concentrated and optionally diafiltered pulse protein solution.

The pulse protein product preferably is an isolate having a protein content of at least about 90 wt %, preferably at least about 100 wt %, (N×6.25) d.b.

The present invention further provides a novel pulse protein product having a protein content of at least about 60 wt %, preferably at least about 90 wt %, more preferably at least about 100 wt % (N×6.25) d.b., and which is water soluble and forms heat stable solutions at acid pH values of less than about 4.4 and is useful for the protein fortification of aqueous systems, including soft drinks and sport drinks, without leading to protein precipitation.

In another aspect of the present invention, there is provided an aqueous solution of the pulse protein product provided herein which is heat stable at a pH of less than about 4.4. The aqueous solution may be a beverage, which may be a clear beverage in which the pulse protein product is completely soluble and transparent or the aqueous solution may be an opaque beverage in which the pulse protein product does or does not contribute to the opacity.

The pulse protein products produced according to the process herein are suitable, not only for protein fortification of acid media, but may be used in a wide variety of conventional applications of protein products, including but not limited to protein fortification of processed foods and beverages, emulsification of oils, as a body former in baked goods and foaming agent in products which entrap gases. In addition, the pulse protein isolates may be formed into protein fibers, useful in meat analogs and may be used as an egg white substitute or extender in food products where egg white is used as a binder. The pulse protein products may also be used in nutritional supplements. Other uses of the pulse protein products are in pet foods, animal feed and in industrial and cosmetic applications and in personal care products.

### GENERAL DESCRIPTION OF INVENTION

The initial step of the process of providing the pulse protein products involves solubilizing pulse protein from a pulse protein source. The pulses to which the invention may be applied include lentils, chickpeas, dry peas and dry beans. The pulse protein source may be pulses or any pulse product or by-product derived from the processing of pulses, such as pulse flour. The pulse protein product recovered from the pulse protein source may be the protein naturally occurring in pulses or the proteinaceous material may be a protein modified by genetic manipulation but possessing characteristic hydrophobic and polar properties of the natural protein.

Protein solubilization from the pulse protein source material is effected most conveniently using calcium chloride solution, although solutions of other calcium salts, may be used. In addition, other alkaline earth metal compounds may be used, such as magnesium salts. Further, extraction of the pulse protein from the pulse protein source may be effected using calcium salt solution in combination with another salt solution, such as sodium chloride. Additionally, extraction of the pulse protein from the pulse protein source may be effected using water or other salt solution, such as sodium chloride, with calcium salt subsequently being added to the aqueous pulse protein solution produced in the extraction step. Precipitate formed upon addition of the calcium salt is removed prior to subsequent processing.

As the concentration of the calcium salt solution increases, the degree of solubilization of protein from the pulse protein source initially increases until a maximum value is achieved. Any subsequent increase in salt concentration does not increase the total protein solubilized. The concentration of calcium salt solution which causes maximum protein solubilization varies depending on the salt concerned. It is usually preferred to utilize a concentration value less than about 1.0 M, and more preferably a value of about 0.10 to about 0.15 M.

In a batch process, the salt solubilization of the protein is effected at a temperature of from about 1° to about 65° C., preferably about 15° C. to about 65° C., more preferably about 20° to about 35° C., preferably accompanied by agitation to decrease the solubilization time, which is usually about 1 to about 60 minutes. It is preferred to effect the solubilization to extract substantially as much protein from the pulse protein source as is practicable, so as to provide an overall high product yield.

In a continuous process, the extraction of the protein from the pulse protein source is carried out in any manner consistent with effecting a continuous extraction of protein from the pulse protein source. In one embodiment, the pulse protein source is continuously mixed with the calcium salt solution and the mixture is conveyed through a pipe or conduit having a length and at a flow rate for a residence time sufficient to effect the desired extraction in accordance with the parameters described herein. In such a continuous procedure, the salt solubilization step is effected rapidly, in a time of up to about 10 minutes, preferably to effect solubilization to extract substantially as much protein from the pulse protein source as is practicable. The solubilization in the continuous procedure is effected at temperatures between about 1° and about 65° C., preferably between about 15° C. and about 65° C., more preferably between about 20° and about 35° C.

The extraction is generally conducted at a pH of about 4.5 to about 11, preferably about 5 to about 7. The pH of the extraction system (pulse protein source and calcium salt solution) may be adjusted to any desired value within the range of about 4.5 to about 11 for use in the extraction step by the use of any convenient food grade acid, usually hydrochloric acid or phosphoric acid, or food grade alkali, usually sodium hydroxide, as required.

The concentration of pulse protein source in the calcium salt solution during the solubilization step may vary widely. Typical concentration values are about 5 to about 15% w/v.

The protein solution resulting from the extraction step generally has a protein concentration of about 5 to about 50 g/L, preferably about 10 to about 50 g/L.

The aqueous calcium salt solution may contain an antioxidant. The antioxidant may be any convenient antioxidant, such as sodium sulfite or ascorbic acid. The quantity of antioxidant employed may vary from about 0.01 to about 1 wt % of the solution, preferably about 0.05 wt %. The antioxidant serves to inhibit oxidation of any phenolics in the protein solution.

The aqueous phase resulting from the extraction step then may be separated from the residual pulse protein source, in any convenient manner, such as by employing a decanter centrifuge, followed by disc centrifugation and/or filtration, to remove residual pulse protein source material. The separation step is generally conducted at the same temperature as the protein solubilization step, but may be conducted at any temperature within the range of about 1° to about 65° C., preferably about 15° to about 65° C., more preferably about 20° to about 35° C. Alternatively, the optional dilution and acidification steps described below may be applied to the mixture of aqueous pulse protein solution and residual pulse protein source, with subsequent removal of the residual pulse protein source material by the separation step described above. The separated residual pulse protein source may be dried for disposal. Alternatively, the separated residual pulse protein source may be processed to recover some residual protein, such as a conventional isoelectric precipitation procedure to recover such residual protein.

The aqueous pulse protein solution may be treated with an adsorbent, such as powdered activated carbon or granulated activated carbon, to remove colour and/or odour compounds. Such adsorbent treatment may be carried out under any convenient conditions, generally at the ambient temperature of the separated aqueous protein solution. For powdered activated carbon, an amount of about 0.025% to about 5% w/v, preferably about 0.05% to about 2% w/v, is employed. The adsorbing agent may be removed from the pulse protein solution by any convenient means, such as by filtration.

The resulting aqueous pulse protein solution may be diluted with water generally with about 0.5 to about 10 volumes, preferably about 0.5 to about 2 volumes, in order to decrease the conductivity of the aqueous pulse protein solution to a value of generally below about 90 mS, preferably about 4 to about 18 mS. Such dilution is usually effected using water, although dilute salt solutions, such as sodium chloride or calcium chloride, having a conductivity up to about 3 mS, may be used.

The water with which the pulse protein solution is mixed generally has the same temperature as the pulse protein solution, but the water may have a temperature of about 1° to about 65° C., preferably about 15° to about 65° C., more preferably about 20° to about 35° C.

The diluted pulse protein solution then is adjusted in pH to a value of about 1.5 to about 4.4, preferably about 2 to about 4, by the addition of any suitable food grade acid, such as hydrochloric acid or phosphoric acid, to result in an acidified aqueous pulse protein solution, preferably a clear acidified aqueous pulse protein solution.

The diluted and acidified pulse protein solution has a conductivity of generally below about 95 mS, preferably about 4 to about 23 mS.

As mentioned above, as an alternative to the earlier separation of the residual pulse protein source, the aqueous pulse protein solution and the residual pulse protein source material, may be optionally diluted and acidified together and then the acidified aqueous pulse protein solution is clarified and separated from the residual pulse protein source material by any convenient technique as discussed above.

The acidified aqueous pulse protein solution may be subjected to a heat treatment to inactivate heat labile anti-nutritional factors, such as trypsin inhibitors, present in such solution as a result of extraction from the pulse protein source material during the extraction step. Such a heating step also provides the additional benefit of reducing the microbial load. Generally, the protein solution is heated to a temperature of about 70° to about 160° C., preferably about 80° to about 120° C., more preferably about 85° to about 95° C., for about 10 seconds to about 60 minutes, preferably about 10 seconds to about 5 minutes, more preferably about 30 seconds to about 5 minutes. The heat treated acidified pulse protein solution then may be cooled for further processing as described below, to a temperature of about 2° to about 65° C., preferably about 20° C. to about 35° C.

If the optionally diluted, acidified and optionally heat treated pulse protein solution is not transparent it may be clarified by any convenient procedure such as filtration or centrifugation.

The resulting acidified aqueous pulse protein solution may be directly dried to produce a pulse protein product. In order to provide a pulse protein product having a decreased impurities content and a reduced salt content, such as a pulse protein isolate, the acidified aqueous pulse protein solution may be processed as described below prior to drying.

The acidified aqueous pulse protein solution may be concentrated to increase the protein concentration thereof while maintaining the ionic strength thereof substantially constant. Such concentration generally is effected to provide a concentrated pulse protein solution having a protein concentration of about 50 to about 300 g/L, preferably about 100 to about 200 g/L.

The concentration step may be effected in any convenient manner consistent with batch or continuous operation, such as by employing any convenient selective membrane technique, such as ultrafiltration or diafiltration, using membranes, such as hollow-fibre membranes or spiral-wound membranes, with a suitable molecular weight cut-off, such as about 3,000 to about 1,000,000 Daltons, preferably about 5,000 to about 100,000 Daltons, having regard to differing membrane materials and configurations, and, for continuous operation, dimensioned to permit the desired degree of concentration as the aqueous protein solution passes through the membranes.

As is well known, ultrafiltration and similar selective membrane techniques permit low molecular weight species to pass therethrough while preventing higher molecular weight species from so doing. The low molecular weight species include not only the ionic species of the salt but also low molecular weight materials extracted from the source material, such as carbohydrates, pigments, low molecular weight proteins and anti-nutritional factors, such as trypsin inhibitors, which are themselves low molecular weight proteins. The molecular weight cut-off of the membrane is usually chosen to ensure retention of a significant proportion of the protein in the solution, while permitting contaminants to pass through having regard to the different membrane materials and configurations.

The concentrated pulse protein solution then may be subjected to a diafiltration step using water or a dilute saline solution. The diafiltration solution may be at its natural pH or at a pH equal to that of the protein solution being diafiltered or at any pH value in between. Such diafiltration may be effected using from about 2 to about 40 volumes of diafiltration solution, preferably about 5 to about 25 volumes of diafiltration solution. In the diafiltration operation, further quantities of contaminants are removed from the aqueous pulse protein solution by passage through the membrane with the permeate. This purifies the aqueous protein solution and may also reduce its viscosity. The diafiltration operation may be effected until no significant further quantities of contaminants and visible colour are present in the permeate or until the retentate has been sufficiently purified so as, when dried, to provide a pulse protein isolate with a protein content of at least about 90 wt % (N×6.25) d.b. Such diafiltration may be effected using the same membrane as for the concentration step. However, if desired, the diafiltration step may be effected using a separate membrane with a different molecular weight cut-off, such as a membrane having a molecular weight cut-off in the range of about 3,000 to about 1,000,000 Daltons, preferably about 5,000 to about 100,000 Daltons, having regard to different membrane materials and configuration.

Alternatively, the diafiltration step may be applied to the acidified aqueous protein solution prior to concentration or to partially concentrated acidified aqueous protein solution. Diafiltration may also be applied at multiple points during the concentration process. When diafiltration is applied prior to concentration or to the partially concentrated solution, the resulting diafiltered solution may then be fully concentrated. The viscosity reduction achieved by diafiltering multiple times as the protein solution is concentrated may allow a higher final, fully concentrated protein concentration to be achieved. This reduces the volume of material to be dried.

The concentration step and the diafiltration step may be effected herein in such a manner that the pulse protein product subsequently recovered contains less than about 90 wt % protein (N×6.25) d.b., such as at least about 60 wt % protein (N×6.25) d.b. By partially concentrating and/or partially diafiltrating the aqueous pulse protein solution, it is possible to only partially remove contaminants. This protein solution may then be dried to provide a pulse protein product with lower levels of purity. The pulse protein product is highly soluble and able to produce protein solutions, preferably clear protein solutions, under acidic conditions.

An antioxidant may be present in the diafiltration medium during at least part of the diafiltration step. The antioxidant may be any convenient antioxidant, such as sodium sulfite or ascorbic acid. The quantity of antioxidant employed in the diafiltration medium depends on the materials employed and may vary from about 0.01 to about 1 wt %, preferably about 0.05 wt %. The antioxidant serves to inhibit the oxidation of any phenolics present in the concentrated pulse protein isolate solution.

The concentration step and the optional diafiltration step may be effected at any convenient temperature, generally about 2° to about 65° C., preferably about 20° to about 35° C., and for the period of time to effect the desired degree of concentration. The temperature and other conditions used to some degree depend upon the membrane equipment used to effect the membrane processing, the desired protein concentration of the solution and the efficiency of the removal of contaminants to the permeate.

As alluded to earlier, pulses contain anti-nutritional trypsin inhibitors. The level of trypsin inhibitor activity in the final pulse protein product can be controlled by the manipulation of various process variables.

As noted above, heat treatment of the acidified aqueous pulse protein solution may be used to inactivate heat-labile trypsin inhibitors. The partially concentrated or fully concentrated acidified pulse protein solution may also be heat treated to inactivate heat labile trypsin inhibitors. When the heat treatment is applied to the partially concentrated acidified pulse protein solution, the resulting heat treated solution may then be additionally concentrated.

In addition, the concentration and/or diafiltration steps may be operated in a manner favorable for removal of trypsin inhibitors in the permeate along with the other contaminants. Removal of the trypsin inhibitors is promoted by using a membrane of larger pore size, such as 30,000 to 1,000,000 Da, operating the membrane at elevated temperatures, such as 30° to 65° C. and employing greater volumes of diafiltration medium, such as 20 to 40 volumes.

Acidifying and membrane processing the pulse protein solution at a lower pH, such as 1.5 to 3, may reduce the trypsin inhibitor activity relative to processing the solution at higher pH, such as 3 to 4.4. When the protein solution is concentrated and diafiltered at the low end of the pH range, it may be desired to raise the pH of the retentate prior to drying. The pH of the concentrated and diafiltered protein solution may be raised to the desired value, for example pH 3, by the addition of any convenient food grade alkali, such as sodium hydroxide.

Further, a reduction in trypsin inhibitor activity may be achieved by exposing pulse materials to reducing agents that disrupt or rearrange the disulfide bonds of the inhibitors. Suitable reducing agents include sodium sulfite, cysteine and N-acetylcysteine.

The addition of such reducing agents may be effected at various stages of the overall process. The reducing agent may be added with the pulse protein source material in the extraction step, may be added to the clarified aqueous pulse protein solution following removal of residual pulse protein source material, may be added to the diafiltered retentate before drying or may be dry blended with the dried pulse protein product. The addition of the reducing agent may be combined with the heat treatment step and membrane processing steps, as described above.

If it is desired to retain active trypsin inhibitors in the concentrated protein solution, this can be achieved by eliminating or reducing the intensity of the heat treatment step, not utilizing reducing agents, operating the concentration and diafiltration steps at the higher end of the pH range, such as 3 to 4.4, utilizing a concentration and diafiltration membrane with a smaller pore size, operating the membrane at lower temperatures and employing fewer volumes of diafiltration medium.

The concentrated and optionally diafiltered aqueous protein solution may be treated with an adsorbent, such as powdered activated carbon or granulated activated carbon, to remove colour and/or odour compounds. Such adsorbent treatment may be carried out under any convenient conditions, generally at the ambient temperature of the concentrated protein solution. For powdered activated carbon, an amount of about 0.025% to about 5% w/v, preferably about 0.05% to about 2% w/v, is employed. The adsorbent may be removed from the pulse protein solution by any convenient means, such as by filtration.

The concentrated and optionally diafiltered aqueous pulse protein solution may be dried by any convenient technique, such as spray drying or freeze drying. A pasteurization step may be effected on the pulse protein solution prior to drying. Such pasteurization may be effected under any desired pasteurization conditions. Generally, the concentrated and optionally diafiltered pulse protein solution is heated to a temperature of about 55° to about 70° C., preferably about 60° to about 65° C., for about 30 seconds to about 60 minutes, preferably about 10 minutes to about 15 minutes. The pasteurized concentrated pulse protein solution then may be cooled for drying, preferably to a temperature of about 25° to about 40° C.

The dry pulse protein product has a protein content greater than about 60 wt %. Preferably, the dry pulse protein product is an isolate with a protein content in excess of about 90 wt % protein, preferably at least about 100 wt %, (N×6.25) d.b.

The pulse protein product produced herein is soluble in an acidic aqueous environment, making the product ideal for incorporation into beverages, both carbonated and uncarbonated, to provide protein fortification thereto. Such beverages have a wide range of acidic pH values, ranging from about 2.5 to about 5. The pulse protein product provided herein may be added to such beverages in any convenient quantity to provide protein fortification to such beverages, for example, at least about 5 g of the pulse protein per serving. The added pulse protein product dissolves in the beverage and the opacity of the beverage is not increased by thermal processing. The pulse protein product may be blended with dried beverage prior to reconstitution of the beverage by dissolution in water. In some cases, modification to the normal formulation of the beverages to tolerate the composition of the invention may be necessary where components present in the beverage may adversely affect the ability of the composition of the invention to remain dissolved in the beverage.

### EXAMPLES

### Example 1

This Example evaluates the protein extractability of lentils, chickpeas and dry peas and the effect of acidification on the clarity of protein solutions resulting from the extraction step.

Dry lentils, chickpeas, yellow split peas and green split peas were purchased in whole form and ground using a Bamix chopper until in the form of a relatively fine powder. The extent of grinding was not controlled by time or particle size. Ground material (10 g) was extracted with 0.15M CaCl₂ (100 ml) for 30 minutes on a magnetic stirrer at room temperature. The extract was separated from the spent material by centrifugation at 10,200 g for 10 minutes and then further clarified by filtration with a 0.45 μm pore size syringe filter. The ground starting material and the clarified extract were tested for protein content using a Leco FP 528 Nitrogen Determinator. The clarity of the extract at full strength and diluted with 1 volume of reverse osmosis purified (RO) water was determined by measuring the absorbance at 600 nm (A600). The full strength and diluted solutions were then adjusted to pH 3 with HCl and the A600 measured again. In this and other Examples where solution clarity was assessed by A600 measurement, water was used to blank the spectrophotometer.

The protein contents and apparent extractabilities determined for each protein source are shown in Table 1.

TABLE 1
Protein content and apparent extractability of protein sources

| protein source | protein content (%) | apparent extractability (%) |
|---|---|---|
| lentil | 24.20 | 47.5 |
| chickpeas | 18.97 | 52.2 |
| yellow split peas | 23.07 | 59.4 |
| green split peas | 22.38 | 64.3 |

As may be seen from the results in Table 1, the apparent extractability of all the protein sources was quite good.

Clarity of the full strength and diluted extract samples before and after acidification are shown in Table 2.

TABLE 2
Effect of acidification on clarity of diluted and undiluted extract samples -
calcium chloride extraction

| | undiluted | | | | diluted | | | |
|---|---|---|---|---|---|---|---|---|
| | initial | initial | final | final | initial | initial | final | final |
| sample | pH | A600 | pH | A600 | pH | A600 | pH | A600 |
| lentils | 5.22 | 0.093 | 3.04 | 0.253 | 5.30 | 1.196 | 2.96 | 0.037 |
| chickpeas | 5.15 | 0.189 | 3.07 | 0.228 | 5.25 | 2.714 | 2.79 | 0.099 |
| yellow split | 5.21 | 0.250 | 3.14 | 0.828 | 5.28 | 2.334 | 3.11 | 0.250 |
| peas | | | | | | | | |
| green split peas | 5.23 | 0.288 | 3.18 | 0.577 | 5.31 | 2.248 | 2.97 | 0.161 |

As may be seen from the results of Table 2, full strength extract solutions from lentil, chickpea and split peas were clear to slightly hazy. Acidification without dilution increased the haze level in the samples. Dilution of the filtered extract with an equal volume of water resulted in notable precipitation and a corresponding increase in the A600 value. Acidification of the diluted solution largely re-solubilized the precipitate and resulted in a clear solution for lentils and chickpeas and a slightly hazy solution for the yellow and green split peas.

### Example 2

This Example contains an evaluation of the clarity of acidified, diluted or undiluted green split pea extracts with water and sodium chloride replacing the calcium chloride solution of Example 1 as the extraction solution.

Dry green split peas were purchased in whole form and ground to a fine powder using a KitchenAid mixer grinder attachment. The extent of grinding was not controlled by time or particle size. Ground material (10 g) was extracted with 0.15M NaCl (100 ml) or RO water (100 ml) for 30 minutes on a magnetic stirrer at room temperature. The extract was separated from the spent material by centrifugation at 10,200 g for 10 minutes and then further clarified by filtration with a 0.45 μm pore size syringe filter. The clarity of the filtrates at full strength and diluted with 1 volume of RO water was determined by measuring the absorbance at 600 nm. The full strength and diluted solutions were then adjusted to pH 3 with HCl and the A600 measured again.

Clarity of the full strength and diluted extract samples before and after acidification are shown in Table 3.

TABLE 3
Effect of acidification on clarity of diluted and undiluted extract samples -
water and sodium chloride extractions

| | undiluted | | | | diluted | | | |
|---|---|---|---|---|---|---|---|---|
| extraction | initial | initial | final | final | initial | initial | final | final |
| solution | pH | A600 | pH | A600 | pH | A600 | pH | A600 |
| water | 6.56 | 0.113 | 3.14 | >3.0 | 6.62 | 0.050 | 3.00 | 2.647 |
| 0.15M NaCl | 6.19 | 0.021 | 2.96 | >3.0 | 6.28 | 0.870 | 2.87 | 2.851 |

As may be seen from the results in Table 3, extracts prepared with water or sodium chloride solution were very cloudy when acidified regardless of whether a dilution step was employed.

### Example 3

This Example evaluates the protein extractability of several types of dry beans and the effect of acidification on the clarity of protein solutions resulting from the extraction step.

Pinto beans, small white beans, small red beans, romano beans, great northern beans and lima beans were purchased in whole, dry form and ground using a Bamix chopper until in the form of a relatively fine powder. The extent of grinding was not controlled by time or particle size. Black bean flour was also purchased. Ground material or flour (10 g) was extracted with 0.15M CaCl₂ (100 ml) for 30 minutes on a magnetic stirrer at room temperature. The extract was separated from the spent material by centrifugation at 10,200 g for 10 minutes and then further clarified by filtration with a 0.45 μm pore size syringe filter. The ground starting material or flour and the clarified extract were tested for protein content using a Leco FP 528 Nitrogen Determinator. The clarity of the extract at full strength and diluted with 1 volume of RO water was determined by measuring the absorbance at 600 nm. The full strength and diluted solutions were then adjusted to pH 3 with HCl and the A600 measured again.

The protein contents and apparent extractabilities determined for each type of dry bean are shown in Table 4.

TABLE 4
Protein content and apparent extractability of various dry beans

| type of bean | protein content (%) | apparent extractability (%) |
|---|---|---|
| black bean | 24.00 | 77.9 |
| pinto bean | 21.45 | 66.2 |
| small white bean | 24.41 | 63.5 |
| small red bean | 20.18 | 76.8 |
| romano bean | 18.07 | 86.9 |
| great northern bean | 21.77 | 85.9 |
| lima bean | 21.43 | 71.9 |

As may be seen from the results in Table 4, the protein in all of the types of beans was readily extracted.

Clarity of the full strength and diluted extract samples before and after acidification are shown in Table 5.

TABLE 5
Effect of acidification on clarity of diluted and undiluted extract samples -
calcium chloride extraction

| | undiluted | | | | diluted 1 + 1 | | | |
|---|---|---|---|---|---|---|---|---|
| | initial | initial | final | final | initial | initial | final | final |
| sample | pH | A600 | pH | A600 | pH | A600 | pH | A600 |
| black bean | 4.69 | 0.100 | 2.99 | 0.154 | 4.76 | 0.025 | 3.15 | 0.031 |
| pinto bean | 5.08 | 0.014 | 3.02 | 0.072 | 5.34 | 0.003 | 3.00 | 0.017 |
| small white | 5.08 | 0.026 | 3.03 | 0.092 | 5.23 | 0.022 | 3.03 | 0.019 |
| bean | | | | | | | | |
| small red bean | 5.06 | 0.028 | 3.07 | 0.093 | 5.33 | 0.014 | 2.97 | 0.021 |
| romano bean | 4.96 | n.d. | 3.07 | 0.023 | 5.21 | 0.005 | 2.86 | 0.008 |
| gr. northern | 4.93 | 0.026 | 3.10 | 0.045 | 5.16 | 0.008 | 3.11 | 0.013 |
| bean | | | | | | | | |
| lima bean | 5.13 | n.d. | 3.07 | 0.089 | 5.37 | 0.020 | 3.04 | 0.013 |

n.d. = not determined

As may be seen from the results of Table 5, full strength extract solutions from all of the beans were quite clear. Acidification without dilution slightly increased the haze level in the samples but they remained quite clear. Dilution of the filtered extract with an equal volume of water did not result in the formation of any precipitate. This is in contrast to the precipitation seen upon dilution for the pulses tested in Example 1. The diluted bean protein solutions stayed clear when acidified.

### Example 4

This Example contains an evaluation of the clarity of acidified, diluted or undiluted small white bean extracts with water and sodium chloride replacing the calcium chloride solution of Example 3 as the extraction solution.

Dry small white beans were purchased in whole form and ground to a fine powder using a Bamix chopper. The extent of grinding was not controlled by time or particle size. Ground material (10 g) was extracted with 0.15M NaCl (100 ml) or RO water (100 ml) for 30 minutes on a magnetic stirrer at room temperature. The extract was separated from the spent material by centrifugation at 10,200 g for 10 minutes and then further clarified by filtration with a 0.45 μm pore size syringe filter. The protein content of the filtrates was determined using a Leco FP528 Nitrogen Determinator. The clarity of the extracts at full strength and diluted with 1 volume of RO water was determined by measuring the absorbance at 600 nm. The full strength and diluted solutions were then adjusted to pH 3 with HCl and the A600 measured again.

Extraction with water and sodium chloride solution provided apparent extractabilities of 45.9% and 61.5% respectively. Clarity of the full strength and diluted extract samples before and after acidification are shown in Table 6.

TABLE 6
Effect of acidification on clarity of diluted and undiluted extract samples -
water and sodium chloride extractions

| | undiluted | | | | diluted | | | |
|---|---|---|---|---|---|---|---|---|
| extraction | initial | initial | final | final | initial | initial | final | final |
| solution | pH | A600 | pH | A600 | pH | A600 | pH | A600 |
| water | 6.48 | 0.079 | 2.95 | >3.0 | 6.51 | 0.051 | 3.03 | 2.771 |
| 0.15M NaCl | 6.13 | 0.116 | 3.01 | >3.0 | 6.22 | 0.212 | 3.02 | >3.0 |

As may be seen from the results in Table 6, extracts prepared with water or sodium chloride solution were very cloudy when acidified regardless of whether a dilution step was employed.

### Example 5

This Example illustrates the production of green pea protein isolate at benchtop scale.

180 g of dry green split peas were finely ground using a KitchenAid mixer grinder attachment. 150 g of finely ground green split pea flour was combined with 1,000 ml of 0.15 M CaCl₂ solution at ambient temperature and agitated for 30 minutes to provide an aqueous protein solution. The residual solids were removed and the resulting protein solution was clarified by centrifugation and filtration to produce a filtered protein solution having a protein content of 1.83% by weight. 655 ml of the filtered protein solution was added to 655 ml of RO water and the pH of the sample lowered to 3.03 with HCl solution.

The diluted and acidified protein extract solution was reduced in volume from 1250 ml to 99 ml by concentration on a PES membrane having a molecular weight cutoff of 10,000 Daltons. An aliquot of 96 ml of concentrated protein solution was then diafiltered on the same membrane with 480 ml of RO water. The resulting acidified, diafiltered, concentrated protein solution had a protein content of 7.97% by weight and represented a yield of 65.5 wt % of the initial filtered protein solution that was further processed. The acidified, diafiltered, concentrated protein solution was dried to yield a product found to have a protein content of 95.69% (N×6.25) d.b. The product was termed GP701-01 protein isolate.

8.30 g of GP701-01 was produced. A solution of GP701-01 was prepared by dissolving sufficient protein powder to provide 0.48 g protein in 15 ml RO water and the pH measured with a pH meter and the colour and clarity assessed using a HunterLab Color Quest XE instrument operated in transmission mode. The results are shown in the following Table 7.

TABLE 7
pH and HunterLab scores for solution of GP701-01

| sample | pH | L* | a* | b* | haze |
|---|---|---|---|---|---|
| GP701-01 | 3.17 | 89.46 | 1.10 | 14.98 | 63.3 |

As may be seen from the results in Table 7, the solution of GP701-01 was translucent and had a light colour.

The solution of GP701-01 was heated to 95° C., held at this temperature for 30 seconds and then immediately cooled to room temperature in an ice bath. The clarity was re-measured with the HunterLab instrument and the results are shown in Table 8.

TABLE 8
HunterLab scores for solution of GP701-01 after heat treatment

| sample | L* | a* | b* | haze |
|---|---|---|---|---|
| GP701-01 | 95.56 | −0.06 | 9.65 | 47.0 |

As may be seen from the results in Table 8, heat treatment was found to improve the lightness and reduce the haze level of the solution while making it greener and less yellow. Although the level of haze in the solution was reduced, the protein solution was still translucent rather than transparent.

### Example 6

This Example illustrates the production of green pea protein isolate at benchtop scale but with the filtration step moved to after dilution and acidification of the extract.

180 g of dry green split peas were finely ground using a KitchenAid mixer grinder attachment. 150 g of finely ground green split pea flour was combined with 1,000 ml of 0.15 M CaCl₂ solution at ambient temperature and agitated for 30 minutes to provide an aqueous protein solution. The residual solids were removed by centrifugation to produce a centrate having a protein content of 2.49% by weight. 800 ml of centrate was added to 800 ml of water and the pH of the sample lowered to 3.00 with diluted HCl. The diluted and acidified centrate was further clarified by filtration to provide a clear protein solution with a protein content of 1.26% by weight. By filtering the solution after dilution and acidification, the A600 of the solution before membrane processing in this trial was 0.012, compared to 0.093 for the diluted and acidified filtrate in Example 5.

The filtered protein solution was reduced in volume from 1292 ml to 157 ml by concentration on a PES membrane having a molecular weight cutoff of 10,000 Daltons. An aliquot of 120 ml of concentrated protein solution was then diafiltered on the same membrane with 600 ml of RO water. The resulting acidified, diafiltered, concentrated protein solution had a protein content of 7.70% by weight and represented a yield of 42.5 wt % of the initial centrate that was further processed. The acidified, diafiltered, concentrated protein solution was dried to yield a product found to have a protein content of 94.23% (N×6.25) d.b. The product was termed GP701-02 protein isolate.

8.55 g of GP701-02 was produced. A solution of GP701-02 was prepared by dissolving sufficient protein powder to provide 0.48 g protein in 15 ml of RO water and the pH measured with a pH meter and the colour and clarity assessed using a HunterLab Color Quest XE instrument operated in transmission mode. The results are shown in the following Table 9.

TABLE 9
pH and HunterLab scores for solution of GP701-02

| sample | pH | L* | a* | b* | haze |
|---|---|---|---|---|---|
| GP701-02 | 3.23 | 90.78 | 0.77 | 14.00 | 47.2 |

As may be seen from the results in Table 9, the GP701-02 solution was translucent and had a light colour. The level of haze was lower than that determined for the solution of GP701-01 in Example 5.

The solution of GP701-02 was heated to 95° C., held at this temperature for 30 seconds and then immediately cooled to room temperature in an ice bath. The clarity was then re-measured with the HunterLab and the result is shown in Table 10 below.

TABLE 10
HunterLab scores for solution of GP701-02 after heat treatment

| sample | L* | a* | b* | haze |
|---|---|---|---|---|
| GP701-02 | 96.24 | −0.48 | 9.74 | 2.2 |

As may be seen from the results in Table 10, heat treatment of the GP701-02 solution resulted in an extremely clear solution.

### Example 7

This Example illustrates the production of small white bean protein isolate at benchtop scale.

About 150 g of small white beans were finely ground using a KitchenAid mixer grinder attachment. 120 g of finely ground small white bean flour was combined with 1,000 ml of 0.15 M CaCl₂ solution at ambient temperature and agitated for 30 minutes to provide an aqueous protein solution. The residual solids were removed and the resulting protein solution was clarified by centrifugation and filtration to produce a filtered protein solution having a protein content of 2.02% by weight. 600 ml of the filtered protein solution was added to 600 ml of RO water and the pH of the sample lowered to 3.01 with diluted HCl. Some wispy particulates were visible in the sample after the pH adjustment and these were removed by passing the sample through 25 μm pore size filter paper.

A sample of the diluted and acidified protein extract solution was then reduced in volume from 1110 ml to 82 ml by concentration on a PES membrane having a molecular weight cutoff of 10,000 Daltons. An aliquot of 79 ml of the retentate was then diafiltered on the same membrane with 395 ml of RO water. The resulting acidified, diafiltered, concentrated protein solution had a protein content of 10.37% by weight and represented a yield of 67.6 wt % of the initial filtered protein solution that was further processed. The acidified, diafiltered, concentrated protein solution was dried to yield a product found to have a protein content of 93.75% (N×6.25) d.b. The product was termed SWB701 protein isolate.

8.26 g of SWB701 was produced. A solution of SWB701 was prepared by dissolving sufficient protein powder to provide 0.48 g protein in 15 ml RO water and the pH measured with a pH meter and the colour and clarity assessed using a HunterLab Color Quest XE instrument operated in transmission mode. The results are shown in the following Table 11.

TABLE 11
pH and HunterLab scores for solution of SWB701

| sample | pH | L* | a* | b* | haze |
|---|---|---|---|---|---|
| SWB701 | 3.09 | 97.42 | 0.22 | 5.29 | 73.2 |

As may be seen from the results in Table 11, the solution of SWB701 was translucent and had a light colour.

The solution of SWB701 was heated to 95° C., held at this temperature for 30 seconds and then immediately cooled to room temperature in an ice bath. The clarity was re-measured with the HunterLab instrument and the results are shown in Table 12.

TABLE 12
HunterLab scores for solution of SWB701 after heat treatment

| sample | L* | a* | b* | haze |
|---|---|---|---|---|
| SWB701 | 98.57 | −0.17 | 4.05 | 50.0 |

As may be seen from the results in Table 12, heat treatment was found to improve the lightness and reduce the haze level of the solution while making it greener and less yellow. Although the level of haze in the solution was reduced, the protein solution was still translucent rather than transparent.

### Example 8

This Example contains an evaluation of the solubility in water of the GP701-02 produced by the method of Example 6 and the SWB701 produced by the method of Example 7. Solubility was tested using a modified version of the procedure of Morr et al., J. Food Sci. 50:1715-1718.

Sufficient protein powder to supply 0.5 g of protein was weighed into a beaker and then approximately 45 ml of reverse osmosis (RO) purified water was added. The contents of the beaker were slowly stirred for 60 minutes using a magnetic stirrer. The pH was determined immediately after dispersing the protein and was adjusted to the appropriate level (2, 3, 4, 5, 6 or 7) with diluted NaOH or HCl. A sample was also prepared at natural pH. For the pH adjusted samples, the pH was measured and corrected periodically during the 60 minutes stirring. After the 60 minutes of stirring, the samples were made up to 50 ml total volume with RO water, yielding a 1% w/v protein dispersion. The protein content of the dispersions was measured using a Leco FP528 Nitrogen Determinator. Aliquots of the dispersions were then centrifuged at 7,800 g for 10 minutes, which sedimented insoluble material. The protein content of the supernatant was then determined by Leco analysis.

The solubility of the protein was then calculated using the following equation:

Solubility (%)=(% protein in supernatant/% protein in initial dispersion)×100

The natural pH values of the protein isolates produced in Examples 6 and 7 are shown in the following Table 13:

TABLE 13
Natural pH of samples prepared in water at 1% w/v protein

| sample | Natural pH |
|---|---|
| GP701-02 | 3.23 |
| SWB701 | 3.09 |

The solubility results obtained are set forth in the following Table 14:

TABLE 14
Solubility of products at different pH values

| | Solubility (%) | | | | | | |
|---|---|---|---|---|---|---|---|
| sample | pH 2 | pH 3 | pH 4 | pH 5 | pH 6 | pH 7 | Nat. pH |
| GP701-02 | 100 | 100 | 100 | 31.1 | 35.7 | 37.8 | 100 |
| SWB701 | 95.2 | 95.3 | 100 | 88.8 | 55.4 | 77.5 | 94.0 |

As can be seen from the results of Table 14, both of the 701 products were extremely soluble over the pH range 2 to 4.

### Example 9

This Example contains an evaluation of the clarity in water of the GP701-02 produced by the method of Example 6 and the SWB701 produced by the method of Example 7.

The clarity of the 1% w/v protein dispersions prepared as described in Example 8 was assessed by analyzing the samples on a HunterLab ColorQuest XE instrument operated in transmission mode to provide a percentage haze reading. A lower score indicated greater clarity.

The clarity results are set forth in the following Table 15:

TABLE 15
Clarity of solutions at different pH values as assessed by HunterLab
analysis

| | HunterLab haze reading (%) | | | | | | |
|---|---|---|---|---|---|---|---|
| sample | pH 2 | pH 3 | pH 4 | pH 5 | pH 6 | pH 7 | Nat. pH |
| GP701-02 | 11.9 | 16.3 | 17.4 | 91.8 | 92.1 | 92.0 | 14.0 |
| SWB701 | 0.0 | 38.0 | 64.6 | 91.7 | 92.4 | 82.9 | 43.9 |

As can be seen from the results of Table 15, the solutions of GP701-02 were substantially clear or slightly hazy in the pH range 2 to 4. The solutions of GP701-02 were cloudy at the higher pH values where the solubility was reduced. The solution of SWB701 had no detectable haze at pH 2, but was noticeably hazier as the pH increased. Note that the protein solubility was still very high in the pH range 3 to 4 even though the solutions were not clear.

### Example 10

This Example illustrates the production of black bean protein product at benchtop scale.

50 g of black bean flour was combined with 500 ml of 0.15 M CaCl₂ solution at ambient temperature and agitated for 30 minutes to provide an aqueous protein solution. The residual solids were removed and the resulting protein solution was clarified by centrifugation and filtration to produce a filtered protein solution having a protein content of 1.18% by weight. 450 ml of the filtered protein solution was added to 450 ml of RO water and the pH of the sample lowered to 3.09 with diluted HCl.

The diluted and acidified protein extract solution was then reduced in volume from 900 ml to 50 ml by concentration on a PES membrane having a molecular weight cutoff of 10,000 Daltons. An aliquot of 40 ml of the retentate was then diafiltered on the same membrane with 200 ml of RO water. The resulting acidified, diafiltered, concentrated protein solution had a protein content of 6.23% by weight and represented a yield of approximately 46.9 wt % of the initial filtered protein solution that was further processed. The acidified, diafiltered, concentrated protein solution was dried to yield a product found to have a protein content of 86.33% (N×6.25) d.b. The product was termed BB701.

2.19 g of BB701 was produced. A solution of BB701 was prepared by dissolving sufficient protein powder to provide 0.48 g protein in 15 ml of RO water and the pH measured with a pH meter and the colour and clarity assessed using a HunterLab Color Quest XE instrument operated in transmission mode. The results are shown in the following Table 16.

TABLE 16
pH and HunterLab scores for solution of BB701

| sample | pH | L* | a* | b* | haze |
|---|---|---|---|---|---|
| BB701 | 3.14 | 95.20 | 0.88 | 8.22 | 54.6 |

As may be seen from the results in Table 16, the solution of BB701 was translucent and had a light colour.

The solution of BB701 was heated to 95° C., held at this temperature for 30 seconds and then immediately cooled to room temperature in an ice bath. The clarity was re-measured with the HunterLab instrument and the results are shown in Table 17.

TABLE 17
HunterLab scores for solution of BB701 after heat treatment

| sample | L* | a* | b* | haze |
|---|---|---|---|---|
| BB701 | 95.89 | 0.54 | 7.81 | 25.2 |

As may be seen from the results in Table 17, heat treatment was found to improve the lightness and reduce the haze level of the solution while making it less red and less yellow. Although the level of haze in the solution was reduced, the protein solution was still hazy rather than transparent.

### Example 11

This Example illustrates the production of yellow pea protein isolate at pilot scale.

20 kg of yellow split pea flour was combined with 200 L of 0.15 M CaCl₂ solution at ambient temperature and agitated for 30 minutes to provide an aqueous protein solution. The residual solids were removed by centrifugation to produce a centrate having a protein content of 1.53% by weight. 180.4 L of centrate was added to 231.1 L of RO water and the pH of the sample lowered to about 3 with diluted HCl. The diluted and acidified centrate was further clarified by filtration to provide a clear protein solution with a protein content of 0.57% by weight and having a pH of 2.93.

The filtered protein solution was reduced in volume from 431 L to 28 L by concentration on a PES membrane, having a molecular weight cutoff of 100,000 Daltons, operated at a temperature of about 30° C. At this point the acidified protein solution, with a protein content of 6.35% by weight, was diafiltered with 252 L of RO water, with the diafiltration operation conducted at about 30° C. The resulting diafiltered solution was then further concentrated to provide 21 kg of acidified, diafiltered, concentrated protein solution with a protein content of 7.62% by weight, which represented a yield of 58.0 wt % of the initial centrate that was further processed. The acidified, diafiltered, concentrated protein solution was dried to yield a product found to have a protein content of 103.27 wt % (N×6.25) d.b. The product was termed YP01-D11-11A YP701 protein isolate.

### Example 12

This Example contains an evaluation of the protein and phytic acid content as well as the trypsin inhibitor activity of the yellow pea protein isolate produced by the method of Example 11 and a commercial yellow pea protein product called Propulse (Nutripea, Portage la Prairie, MB).

Protein content was determined by a combustion method using a LecoTruSpec N Nitrogen Determinator. Phytic acid content was determined using the method of Latta and Eskin (J. Agric. Food Chem., 28: 1313-1315). Trypsin inhibitor activity (TIA) was determined using AOCS method Ba 12-75 for the commercial protein sample and a modified version of this method for the YP701 product, which has a lower pH when rehydrated.

The results obtained are set forth in the following Table 18:

TABLE 18
Protein content, phytic acid content and trypsin
inhibitor activity of protein products

| | | % protein | % phytic | TIA (TIU/mg |
|---|---|---|---|---|
| | | (N x 6.25) | acid | protein |
| Batch | Product | d.b. | d.b. | (N x 6.25)) |
| YP01-D11-11A | YP701 | 103.27 | 0.27 | 4.6 |
| | Propulse | 82.33 | 2.72 | 3.3 |

As may be seen from the results presented in Table 19, the YP701 was very high in protein and low in phytic acid compared to the commercial product. The trypsin inhibitor activity in both products was very low.

### Example 13

This Example contains an evaluation of the dry colour and colour in solution of the yellow pea protein isolate produced by the method of Example 11 and a commercial yellow pea protein product called Propulse (Nutripea, Portage la Prairie, MB).

The colour of the dry powders was assessed using a HunterLab ColorQuest XE instrument in reflectance mode. The colour values are set forth in the following Table 19:

TABLE 19
HunterLab scores for dry protein products

| Sample | L* | a* | b* |
|---|---|---|---|
| YP01-D11-11A YP701 | 86.27 | 2.21 | 9.73 |
| Propulse | 82.39 | 3.29 | 20.94 |

As may be seen from Table 19, the YP01-D11-11A YP701 powder was lighter, less red and less yellow in colour compared to the commercial yellow pea protein product.

Solutions of the yellow pea protein products were prepared by dissolving sufficient protein powder to supply 0.48 g of protein in 15 ml of RO water. The pH of the solutions was measured with a pH meter and the colour and clarity assessed using a HunterLab Color Quest XE instrument operated in transmission mode. Hydrochloric acid solution was added to the Propulse sample to lower the pH to 3 and then the measurement repeated. The results are shown in the following Table 20.

TABLE 20
pH and HunterLab scores for solutions
of yellow pea protein products

| sample | pH | L* | a* | b* | haze |
|---|---|---|---|---|---|
| YP01-D11-11A YP701 | 3.45 | 93.97 | 0.54 | 12.70 | 5.0 |
| Propulse | 6.15 | 35.33 | 12.61 | 48.79 | 96.6 |
| Propulse (pH adjusted) | 3.00 | 37.83 | 11.55 | 47.87 | 96.9 |

As may be seen from the results in Table 20, the YP01-D11-11A YP701 solution was transparent while the Propulse solution was very cloudy regardless of pH. The YP01-D11-11A YP701 solution was also much lighter, less red and less yellow than the Propulse solution regardless of its pH.

### Example 14

This Example contains an evaluation of the heat stability in water of the yellow pea protein isolate produced by the method of Example 11 and a commercial yellow pea protein product called Propulse (Nutripea, Portage la Prairie, MB).

2% w/v protein solutions of YP01-D11-11A YP701 and Propulse were prepared in RO water. The natural pH of the solutions was determined with a pH meter. The samples were each split into two portions and the pH of one portion was lowered to 3.00 with HCl solution. The clarity of the control and pH adjusted solutions was assessed by haze measurement with the HunterLab Color Quest XE instrument operated in transmission mode. The solutions were then heated to 95° C., held at this temperature for 30 seconds and then immediately cooled to room temperature in an ice bath. The clarity of the heat treated solutions was then measured again.

The clarity of the protein solutions before and after heating is set forth in the following Table 21:

TABLE 21
Effect of heat treatment on clarity of 2% w/v protein
solutions of yellow pea protein products

| | | haze before heat | haze after heat |
|---|---|---|---|
| sample | pH | treatment (%) | treatment (%) |
| YP01-D11-11A YP701 | 3.70 | 3.6 | 1.4 |
| YP01-D11-11A YP701 | 3.00 | 2.8 | 1.3 |
| (pH adjusted) | | | |
| Propulse | 6.24 | 96.1 | 96.4 |
| Propulse (pH adjusted) | 3.00 | 96.6 | 96.6 |

As can be seen from the results in Table 21, the solutions of YP01-D11-11A YP701 were transparent before and after heating at both pH levels. The solutions of Propulse were highly cloudy before and after heating at both pH levels.

### Example 15

This Example contains an evaluation of the solubility in water of the yellow pea protein isolate produced by the method of Example 11 and a commercial yellow pea protein product called Propulse (Nutripea, Portage la Prairie, MB). Solubility was tested based on protein solubility (termed protein method, a modified version of the procedure of Morr et al., J. Food Sci. 50:1715-1718) and total product solubility (termed pellet method).

Sufficient protein powder to supply 0.5 g of protein was weighed into a beaker and then a small amount of reverse osmosis (RO) purified water was added and the mixture stirred until a smooth paste formed. Additional water was then added to bring the volume to approximately 45 ml. The contents of the beaker were then slowly stirred for 60 minutes using a magnetic stirrer. The pH was determined immediately after dispersing the protein and was adjusted to the appropriate level (2, 3, 4, 5, 6 or 7) with diluted NaOH or HCI. A sample was also prepared at natural pH. For the pH adjusted samples, the pH was measured and corrected periodically during the 60 minutes stirring. After the 60 minutes of stirring, the samples were made up to 50 ml total volume with RO water, yielding a 1% w/v protein dispersion. The protein content of the dispersions was measured using a Leco TruSpec N Nitrogen Determinator. Aliquots (20 ml) of the dispersions were then transferred to pre-weighed centrifuge tubes that had been dried overnight in a 100° C. oven then cooled in a desiccator and the tubes capped. The samples were centrifuged at 7,800 g for 10 minutes, which sedimented insoluble material and yielded a clear supernatant. The protein content of the supernatant was measured by Leco analysis and then the supernatant and the tube lids were discarded and the pellet material dried overnight in an oven set at 100° C. The next morning the tubes were transferred to a desiccator and allowed to cool. The weight of dry pellet material was recorded. The dry weight of the initial protein powder was calculated by multiplying the weight of powder used by a factor of ((100−moisture content of the powder (%))/100). Solubility of the product was then calculated two different ways:

Solubility (protein method) (%)=(% protein in supernatant/% protein in initial dispersion)×100 1)

Solubility (pellet method) (%)=(1−(weight dry insoluble pellet material/((weight of 20 ml of dispersion/weight of 50 ml of dispersion)×initial weight dry protein powder)))×100 2)

The natural pH values of the protein isolate produced in Example 11 and the commercial yellow pea protein product in water (1% protein) are shown in Table 22:

TABLE 22
Natural pH of YP01-D11-11A YP701 and Propulse
solutions prepared in water at 1% protein

| Batch | Product | Natural pH |
|---|---|---|
| YP01-D11-11A | YP701 | 3.56 |
| | Propulse | 6.15 |

The solubility results obtained are set forth in the following Tables 23 and 24:

TABLE 23
Solubility of products at different pH values based on protein method

| | | Solubility (protein method) (%) | | | | | | |
|---|---|---|---|---|---|---|---|---|
| Batch | Product | pH 2 | pH 3 | pH 4 | pH 5 | pH 6 | pH 7 | Nat. pH |
| YP01- | YP701 | 98.2 | 99.1 | 99.5 | 50.9 | 20.4 | 39.3 | 100 |
| D11- | | | | | | | | |
| 11A | | | | | | | | |
| | Propulse | 14.9 | 3.6 | 2.6 | 5.3 | 10.3 | 7.0 | 8.0 |

TABLE 24
Solubility of products at different pH values based on pellet method

| | | Solubility (pellet method) (%) | | | | | | |
|---|---|---|---|---|---|---|---|---|
| Batch | Product | pH 2 | pH 3 | pH 4 | pH 5 | pH 6 | pH 7 | Nat. pH |
| YP01- | YP701 | 99.6 | 99.3 | 99.1 | 74.7 | 34.7 | 39.1 | 99.0 |
| D11- | | | | | | | | |
| 11A | | | | | | | | |
| | Propulse | 15.5 | 14.7 | 11.6 | 12.1 | 16.4 | 18.0 | 16.5 |

As can be seen from the results presented in Table 23 and 24, the YP01-D11-11A YP701 was highly soluble in the pH range of 2 to 4 and less soluble at higher pH values. The Propulse was very poorly soluble at all pH values tested.

### Example 16

This Example contains an evaluation of the clarity in water of the yellow pea protein isolate produced by the method of Example 11 and a commercial yellow pea protein product called Propulse (Nutripea, Portage la Prairie, MB).

The clarity of the 1% w/v protein solutions prepared as described in Example 15 was assessed by measuring the absorbance at 600 nm, with a lower absorbance score indicating greater clarity. Analysis of the samples on a HunterLab ColorQuest XE instrument in transmission mode also provided a percentage haze reading, another measure of clarity.

The clarity results are set forth in the following Tables 25 and 26:

TABLE 25
Clarity of protein solutions at different pH values as assessed by A600

| | | A600 | | | | | | |
|---|---|---|---|---|---|---|---|---|
| Batch | Product | pH 2 | pH 3 | pH 4 | pH 5 | pH 6 | pH 7 | Nat. pH |
| YP01- | YP701 | 0.012 | 0.015 | 0.024 | 1.962 | 2.829 | 2.557 | 0.021 |
| D11- | | | | | | | | |
| 11A | | | | | | | | |
| | Propulse | 2.576 | 2.579 | 2.693 | 2.685 | 2.588 | 2.560 | 2.590 |

TABLE 26
Clarity of protein solutions at different pH values as assessed by
HunterLab haze analysis

| | | HunterLab haze reading (%) | | | | | | |
|---|---|---|---|---|---|---|---|---|
| Batch | Product | pH 2 | pH 3 | pH 4 | pH 5 | pH 6 | pH 7 | Nat. pH |
| YP01- | YP701 | 0.0 | 0.1 | 1.1 | 95.9 | 96.7 | 96.4 | 0.7 |
| D11- | | | | | | | | |
| 11A | | | | | | | | |
| | Propulse | 96.2 | 96.3 | 96.7 | 96.7 | 96.2 | 96.4 | 96.4 |

As can be seen from the results of Tables 25 and 26, the solutions of YP01-D11-11A YP701 were transparent in the range of pH 2 to 4 but very cloudy at higher pH values. The solutions of Propulse were very cloudy regardless of pH.

### Example 17

This Example contains an evaluation of the solubility in a soft drink (Sprite) and sports drink (Orange Gatorade) of the yellow pea protein isolate produced by the method of Example 11 and a commercial yellow pea protein product called Propulse (Nutripea, Portage la Prairie, MB). The solubility was determined with the protein added to the beverages with no pH correction and again with the pH of the protein fortified beverages adjusted to the level of the original beverages.

When the solubility was assessed with no pH correction, a sufficient amount of protein powder to supply 1 g of protein was weighed into a beaker and a small amount of beverage was added and stirred until a smooth paste formed. Additional beverage was added to bring the volume to 50 ml, and then the solutions were stirred slowly on a magnetic stirrer for 60 minutes to yield a 2% protein w/v dispersion. The protein content of the samples was analyzed using a Leco TruSpec N Nitrogen Determinator then an aliquot of the protein containing beverages was centrifuged at 7,800 g for 10 minutes and the protein content of the supernatant measured.

Solubility (%)=(% protein in supernatant/% protein in initial dispersion)×100.

When the solubility was assessed with pH correction, the pH of the soft drink (Sprite) (3.42) and sports drink (Orange Gatorade) (3.11) without protein was measured. A sufficient amount of protein powder to supply 1 g of protein was weighed into a beaker and a small amount of beverage was added and stirred until a smooth paste formed. Additional beverage was added to bring the volume to approximately 45 ml, and then the solutions were stirred slowly on a magnetic stirrer for 60 minutes. The pH of the protein containing beverages was determined immediately after dispersing the protein and was adjusted to the original no-protein pH with HCl or NaOH as necessary. The pH was measured and corrected periodically during the 60 minutes stirring. After the 60 minutes of stirring, the total volume of each solution was brought to 50 ml with additional beverage, yielding a 2% protein w/v dispersion. The protein content of the samples was analyzed using a Leco TruSpec N Nitrogen Determinator then an aliquot of the protein containing beverages was centrifuged at 7,800 g for 10 minutes and the protein content of the supernatant measured.

Solubility (%)=(% protein in supernatant/% protein in initial dispersion)×100

The results obtained are set forth in the following Table 27:

TABLE 27
Solubility of yellow pea protein products in Sprite and Orange Gatorade

| | | no pH correction | | pH correction | |
|---|---|---|---|---|---|
| | | Solubility (%) | Solubility (%) in | Solubility (%) | Solubility (%) in |
| Batch | Product | in Sprite | Orange Gatorade | in Sprite | Orange Gatorade |
| YP01-D11-11A | YP701 | 98.1 | 100 | 96.6 | 100 |
| | Propulse | 3.2 | 4.6 | 5.6 | 7.4 |

As can be seen from the results of Table 27, the YP01-D11-11A YP701 was highly soluble in the Sprite and the Orange Gatorade. As the YP701 is an acidified product, its addition did not significantly alter the pH of the beverages. The Propulse was very poorly soluble in the beverages tested. Addition of Propulse increased the pH of the drinks but the solubility of the protein was not improved by lowering the pH of the drink back to its original no-protein value.

### Example 18

This Example contains an evaluation of the clarity in a soft drink and sports drink of the yellow pea protein isolate produced by the method of Example 11 and a commercial yellow pea protein product called Propulse (Nutripea, Portage la Prairie, MB).

The clarity of the 2% w/v protein dispersions prepared in soft drink (Sprite) and sports drink (Orange Gatorade) in Example 17 were assessed using the A600 and HunterLab haze methods described in Example 16.

The results obtained are set forth in the following Tables 28 and 29:

TABLE 28
A600 readings for yellow pea protein products
in Sprite and Orange Gatorade

| | | no pH correction | | pH correction | |
|---|---|---|---|---|---|
| | | | A600 in | | A600 in |
| | | A600 in | Orange | A600 in | Orange |
| Batch | Product | Sprite | Gatorade | Sprite | Gatorade |
| no protein | | 0.007 | 0.450 | 0.007 | 0.450 |
| YP01-D11- | YP701 | 0.048 | 0.338 | 0.043 | 0.345 |
| 11A | | | | | |
| | Propulse | 2.800 | 2.834 | 2.827 | 2.793 |

TABLE 29
HunterLab haze readings for yellow pea protein
products in Sprite and Orange Gatorade

| | | no pH correction | | pH correction | |
|---|---|---|---|---|---|
| | | Haze (%) | Haze (%) | Haze (%) | Haze (%) |
| | | in | in Orange | in | in Orange |
| Batch | Product | Sprite | Gatorade | Sprite | Gatorade |
| no protein | | 0.0 | 78.6 | 0.0 | 78.6 |
| YP01-D11- | YP701 | 5.7 | 56.7 | 4.9 | 57.7 |
| 11A | | | | | |
| | Propulse | 97.1 | 97.5 | 96.3 | 96.3 |

As can be seen from the results of Tables 28 and 29, the addition of YP01-D11-11A YP701 to the soft drink and sports drink added little or no haziness, while the addition of the Propulse made the drinks very cloudy, even when the pH was corrected.

### SUMMARY OF THE DISCLOSURE

In summary of this disclosure, the present invention provides novel pulse protein products which are completely soluble and form heat stable, preferably transparent, solutions at acid pH and are useful in the protein fortification of aqueous systems, including soft drinks and sport drinks, without leading to protein precipitation. Modifications are possible within the scope of this invention.

What we claim is:

1. A method of producing a pulse protein product having a protein content of at least about 60 wt %, preferably at least about 90 wt %, (N×6.25) on a dry weight basis, which comprises:
(a) extracting a pulse protein source with an aqueous calcium salt solution to cause solubilization of pulse protein from the protein source and to form an aqueous pulse protein solution,
(b) at least partially separating the aqueous pulse protein solution from residual pulse protein source,
(c) optionally diluting the aqueous pulse protein solution,
(d) adjusting the pH of the aqueous pulse protein solution to a pH of about 1.5 to about 4.4 to produce an acidified aqueous pulse protein solution,
(e) optionally clarifying the acidified pulse protein solution if it is not already clear,
(f) alternatively from steps (b) to (e), optionally diluting and then adjusting the pH of the combined aqueous pulse protein solution and residual pulse protein source to a pH of about 1.5 to about 4.4 then separating the acidified aqueous pulse protein solution from residual pulse protein source,
(g) optionally concentrating the aqueous pulse protein solution while maintaining the ionic strength substantially constant by a selective membrane technique,
(h) optionally diafiltering the concentrated pulse protein solution, and
(i) optionally drying the concentrated and optionally diafiltered pulse protein solution.

2. The method of claim 1 wherein said aqueous calcium salt solution is an aqueous calcium chloride solution.

3. The method of claim 2 wherein said aqueous calcium chloride solution has a concentration less than about 1.0 M.

4. The method of claim 3 wherein said concentration is about 0.10 to about 0.15 M.

5. The process of claim 1 wherein said extraction step (a) is effected at a temperature of about 1° to about 65° C., preferably about 15° to about 65° C., more preferably 20° to about 35° C.

6. The process of claim 1 wherein said extraction with aqueous calcium salt solution is conducted at a pH of about 4.5 to about 11.

7. The process of claim 6 wherein said pH is about 5 to about 7.

8. The process of claim 1 wherein said aqueous pulse protein solution has a protein concentration of about 5 to about 50 g/L.

9. The process of claim 8 wherein said protein concentration is about 10 to about 50 g/L.

10. The process of claim 1 wherein said aqueous calcium salt solution contains an antioxidant.

11. The process of claim 1 wherein, following said separation step (b) and prior to said optional dilution step (c) or in step (f) prior to said optional dilution step, said aqueous pulse protein solution is treated with an adsorbent to remove colour and/or odour compounds from the aqueous pulse protein solution.

12. The process of claim 1 wherein said aqueous pulse protein solution is diluted in step (c) or (f) to a conductivity of less than about 90 mS.

13. The process of claim 12 wherein said aqueous pulse protein solution is diluted in step (c) or (f) with about 0.5 to about 10 volumes of aqueous diluent to provide a conductivity of said pulse protein solution of about 4 to about 18 mS.

14. The process of claim 12 wherein said aqueous diluent has a temperature of about 1° to about 65° C.

15. The process of claim 14 wherein said temperature is about 15° to about 65° C.

16. The process of claim 15 wherein said temperature is about 20° to about 35° C.

17. The process of claim 1 wherein said acidified pulse protein solution has a conductivity of less than about 95 mS.

18. The process of claim 17 wherein said conductivity is about 4 to about 23 mS.

19. The process of claim 1 wherein the pH of said aqueous pulse protein solution is adjusted in step (d) or (f) to about pH 2 to about 4.

20. The process of claim 1 wherein the acidified pulse protein solution, is subjected to step (e).

21. The process of claim 1, wherein said acidified aqueous protein solution following step (d) or step (f) is subjected to a heat treatment step to inactivate heat-labile anti-nutritional factors.

22. The process of claim 21 wherein the anti-nutritional factors are heat-labile trypsin inhibitors.

23. The process of claim 21 wherein the heat treatment step also pasteurizes the acidified aqueous protein solution.

24. The process of claim 21, wherein said heat treatment is effected at a temperature of about 70° to about 160° C. for about 10 seconds to about 60 minutes.

25. The process of claim 24 wherein said heat treatment is effected at a temperature of about 80° to about 120° C. for about 10 seconds to about 5 minutes.

26. The process of claim 25 wherein said heat treatment is effected at a temperature of about 85° C. to about 95° C. for about 30 seconds to about 5 minutes.

27. The process of claim 21 wherein the heat treated acidified pulse protein solution is cooled to a temperature of about 2° to about 65° C. for further processing.

28. The process of claim 27 wherein the heat treated acidified pulse protein solution is cooled to a temperature of about 20° to about 35° C. for further processing.

29. The process of claim 21 wherein the heat treated pulse protein solution is subjected to a polishing step.

30. The process of claim 1 wherein said acidified aqueous pulse protein solution is dried to provide a pulse protein product having a protein content of at least about 60 wt % (N×6.25) d.b.

31. The process of claim 1 wherein said acidified aqueous pulse protein solution is subjected to step (g) to produce a concentrated acidified pulse protein solution having a protein concentration of about 50 to about 300 g/L and the concentrated acidified pulse protein solution is optionally subjected to step (h).

32. The process of claim 31 wherein said concentrated acidified pulse protein solution has a protein concentration of about 100 to about 200 g/L.

33. The process of claim 31 wherein said concentration step (g) is effected by ultrafiltration using a membrane having a molecular weight cut-off of about 3,000 to about 1,000,000 Daltons.

34. The process of claim 33 wherein said membrane has a molecular weight cut-off of about 5,000 to about 100,000 Daltons.

35. The process of claim 31 wherein step (h) is effected using water, acidified water, dilute saline or acidified dilute saline on the acidified pulse protein solution before or after partial or complete concentration thereof.

36. The process of claim 35 wherein said diafiltration step (h) is effected using about 2 to about 40 volumes of diafiltration solution.

37. The process of claim 36 wherein said diafiltration step (h) is effected using about 5 to about 25 volumes of diafiltration solution.

38. The process of claim 35 wherein said diafiltration step (h) is effected until no significant further quantities of contaminants or visible colour are present in the permeate.

39. The process of claim 35 wherein said diafiltration step (h) is effected until the retentate has been sufficiently purified so as, when dried, to provide a pulse protein isolate with a protein content of at least about 90 wt % (N×6.25) d.b.

40. The process of claim 35 wherein said diafiltration step (h) is effected using a membrane having a molecular weight cut-off of about 3,000 to about 1,000,000 Daltons.

41. The process of claim 40 wherein said membrane has a molecular weight cut-off of about 5,000 to about 100,000 Daltons.

42. The process of claim 35 wherein an antioxidant is present in the diafiltration medium during at least part of the diafiltration step (h).

43. The process of claim 31 wherein said concentration step (g) and optional diafiltration step (h) are carried out at a temperature of about 2° to about 65° C.

44. The process of claim 43 wherein said temperature is about 20° to about 35° C.

45. The process of claim 31 wherein the partially concentrated or concentrated and optionally diafiltered acidified pulse protein solution is subjected to a heat treatment step to inactivate heat-labile anti-nutritional factors, including heat-labile trypsin inhibitors.

46. The process of claim 45 wherein said heat treatment is effected at a temperature of about 70° to about 160° C. for about 10 seconds to about 60 minutes, preferably a temperature of about 80° C. to about 120° C. for about 10 seconds to about 5 minutes, and more preferably about 85° C. to about 95° C. for about 30 seconds to about 5 minutes.

47. The process of claim 46 wherein the heat treated pulse protein solution is cooled to a temperature of about 2° to about 65° C., preferably about 20° to about 35° C. for further processing.

48. The process of claim 1 wherein said acidified aqueous pulse protein solution is subjected to steps (g) and (h) to produce a concentrated and/or diafiltered acidified pulse protein solution which, when dried, provides a pulse protein product having a protein concentration of at least about 60 wt % (N×6.25) d.b.

49. The process of claim 31 wherein said concentrated and optionally diafiltered acidified pulse protein solution is treated with an adsorbent to remove colour and/or odour compounds.

50. The process of claim 31 wherein said concentrated and optionally diafiltered acidified pulse protein solution is pasteurized prior to drying.

51. The process of claim 50 wherein said pasteurization step is effected at a temperature of about 55° to about 70° C. for about 30 seconds to about 60 minutes.

52. The process of claim 51 wherein said pasteurization step is effected at a temperature of about 60° to about 65° C. for about 10 to about 15 minutes.

53. The process of claim 39 wherein said concentrated and diafiltered acidified pulse protein solution is subjected to step (i) to provide a pulse protein isolate having a protein content of at least about 90 wt % (N×6.25) d.b.

54. The process of claim 53 wherein said pulse protein isolate has a protein content of at least about 100 wt % (N×6.25) d.b.

55. The process of claim 31 wherein the concentration and/or optional diafiltration step are operated in a manner favourable to the removal of trypsin inhibitors.

56. The process of claim 1 wherein a reducing agent is present during the extraction step (a) to disrupt or rearrange the disulfide bonds of trypsin inhibitors to achieve a reduction in trypsin inhibitor activity.

57. The process of claim 31 wherein a reducing agent is present during the concentration and/or optional diafiltration steps (g) and (h) to disrupt or rearrange the disulfide bonds of trypsin inhibitors to achieve a reduction in trypsin inhibitor activity.

58. The process of claim 48 wherein a reducing agent is added to the concentrated and optionally diafiltered pulse protein solution prior to the drying step (i) and/or the dried pulse protein product to disrupt or rearrange the disulfide bonds of trypsin inhibitors to achieve a reduction in trypsin inhibitor activity.

59. A pulse protein product having a protein content of at least about 60 wt % (N×6.25) d.b. which is water soluble and produces heat stable solutions at acid pH values of less than about 4.4.

60. The pulse protein product of claim 59 having a protein content of at least about 90 wt % (N×6.25) d.b.

61. The protein product of claim 59 having a protein content of at least about 100 wt % (N×6.25) d.b.

62. The protein product of claim 59 which is blended with water-soluble powdered materials for the production of aqueous solutions of the blend.

63. The blend of claim 62 which is a powdered beverage.

64. An aqueous solution of the pulse protein product of claim 59 which is heat stable at a pH of less than about 4.4.

65. The aqueous solution of claim 64 which is a beverage.
